(19) United States
(12) Patent Application Publication
(10) Pub. No.: US 2022/0287927 A1
(43) Pub. Date: Sep. 15, 2022

(54) Systems and methods to provide towelette units.

(71) Applicant: Sweetums, Inc., Carlsbad, CA (US)

(72) Inventor: Alisa Brady, Carlsbad, CA (US)

(21) Appl. No.: 17/829,496

(22) Filed: Jun. 1, 2022

Related U.S. Application Data
(63) Continuation-in-part of application No. 17/129,046, filed on Dec. 21, 2020.

(51) Int. Cl. A61K 8/02; A61K 8/18

(52) U.S. Cl. CPC A61K 8/0208; A61K 8/18

(57) ABSTRACT

A moistening wipe is disclosed. The wipe includes a moistening agent and a flavoring agent. The moisturizing and flavoring agents are deposited on a material. The flavoring agent imparts a pleasant taste to the surface upon which the wipe is used.

### CROSS REFERENCE TO RELATED APPLICATIONS

This application is a Continuation-In-Part (CIP) from U.S. Utility patent application Ser. No. 17/129,046, filed Dec. 21, 2020, the contents of which are incorporated by reference.

### STATEMENT REGARDING FEDERALLY SPONSORED RESEARCH OR DEVELOPMENT

This invention was not federally sponsored.

### BACKGROUND OF THE INVENTION

Field of the invention: This invention relates to the general field of flavored items, and more specifically to lubricating wipes and solutions, flavoring and perfume wipes and solutions.

### Background

Male and female genitalia can be a source of pleasure for individuals and their partners. There are many methods for stimulation and sexual arousal. Among these methods are licking or sucking the genitalia. While this is pleasurable and sexually arousing to the recipient it may be difficult for the one engaging in the licking and sucking because of odors or tastes. Refreshing the genitals and the surrounding area can alleviate the odors and can remove any surface germs, thus providing a more pleasurable environment for the partner whose mouth is on and near the genitals. Additionally, depositing pleasant tasting substances in these areas will improve the experience.

### SUMMARY

In a first aspect, the disclosure provides wipes for lubricating which include flavors and perfumes to make them more desirable to taste.

In a second aspect the disclosure provides a method for imparting flavor and smell to wipes used for lubricating and refreshing skin.

Further aspects and embodiments are provided in the foregoing drawings, detailed description, and claims.

### DETAILED DESCRIPTION

The following description recites various aspects and embodiments of the inventions disclosed herein. No particular invention is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly indicates otherwise. For example, reference to “a substituent” encompasses a single substituent as well as two or more substituents, and the like.

As used herein, “for example,” “for instance.” “such as,” or “including” are meant to introduce examples that further clarify more general subject matter. Unless otherwise expressly indicated, such examples are provided only to aid in understanding embodiments illustrated in the present disclosure and are not meant to be limiting in any fashion. Nor do these phrases indicate any kind of preference for the disclosed embodiment.

As used herein “sweeteners” is meant to refer to compounds both natural and artificial that impart sweetness. Non limited-examples of sweeteners includes common saccharide sweeteners, e.g., sucrose, fructose (e.g., D-fructose), glucose (e.g., D-glucose); sweetener compositions comprising natural sugars, such as stevia (all types and grades), corn syrup (including high fructose corn syrup) or other syrups or sweetener concentrates derived from natural fruit and vegetable sources; semisynthetic “sugar alcohol” sweeteners such as erythritol, isomalt, lactitol, mannitol, sorbitol, xylitol, maltodextrin, glycerol, threitol, arabitol, ribitol, and dulcitol; artificial sweeteners such as miraculin, aspartame, superaspartame, saccharin, saccharin-sodium salt, acesulfame-K, cyclamate, sodium cyclamate, and alitame; other sweeteners such as trehalose, melizitose, melibiose, raffinose, palatinose, lactulose, cyclamic acid, mogroside, tagatose (e.g., D-tagatose), maltose, galactose (e.g., D-galactose), L-rhamnose, D-sorbose, maunose (e.g., D-maunose), lactose, L-arabinose, D-ribose, D-glyceraldehyde, curculin, brazzein, mogroside, Neohesperidin dihydrochalcone (NHDC), neotame and other aspartame derivatives, D-tryptophan, D-leucine, D-threonine, glycine, D-asparagine, D-phenylalanine, L-proline, maltitol, hydrogenated glucose syrup (HGS), magap, sucralose, lugduname, sucrononate, sucrooctate, monatin, phyllodulcin, hydrogenated starch hydrolyzate (HSH), stevioside, rebaudioside A, rebaudioside D, rebadioside M, and other sweet Stevia based glycosides, lo han guo, thaumatin, monellin, carrelameand and other guanidine-based sweeteners.

As used herein “flavoring or perfuming co-ingredient” is meant to refer to a compound, which is used in flavoring or perfuming preparations or compositions to impart a hedonic effect. In other words, such an ingredient, to be considered as being a flavoring or perfuming one, must be recognized by a person skilled in the art as being able to impart or modify in a positive or pleasant way the taste or the odor of a composition, and not just as having a taste or an odor.

The nature and type of the flavoring or perfuming co-ingredients present in the composition do not warrant a more detailed description here, the skilled person being able to select them on the basis of its general knowledge and according to intended use or application and the desired organoleptic effect. In general terms, these flavoring or perfuming co-ingredients belong to chemical classes as varied as alcohols, aldehydes, ketones, esters, ethers, acetates, nitriles, terpenoids, nitrogenous or sulphurous heterocyclic compounds and essential oils, and said flavoring or perfuming co-ingredients can be of natural or synthetic origin. Many of these co-ingredients are in any case listed in reference texts such as the book by S. Arctander, Perfume and Flavor Chemicals, 1969, Montclair, N.J., USA, or its more recent versions, or in other works of a similar nature, as well as in the abundant patent literature in the field of flavor and perfumery. It is also understood that said co-ingredients may also be compounds known to release in a controlled manner various types of flavoring or perfuming compounds.

As used herein “flavor or perfumery carrier” is meant to refer to a material which is substantially neutral from a flavor or perfumery point of view, insofar as it does not significantly alter the organoleptic properties of flavoring ingredients or of perfumery ingredients. The carrier may be a liquid or a solid. Suitable liquid carriers include, for instance, an emulsifying system, i.e., a solvent and a surfactant system, or a solvent commonly used in flavors or perfumery. A detailed description of the nature and type of solvents commonly used in flavor or perfumery cannot be exhaustive. Suitable solvents used in flavor include, for instance, propylene glycol, triacetine, caprylic/capric triglyceride (Neobee®), triethyl citrate, benzylic alcohol, ethanol, vegetable oils such as Linseed oil, sunflower oil or coconut oil, glycerol. One can cite as non-limiting examples of perfumery solvents, solvents such as butylene or propylene glycol, glycerol, diprop yleneglycol and its monoether, I,2,3-propanetriyl triacetate, dimethyl glutarate, dimethyl adipate I,3-diacetyloxypropan-2-yl acetate, diethyl phthalate, isopropyl myristate, benzyl benzoate, benzyl alcohol, 2-(2-ethoxyethoxy)-I-ethano, tri-ethyl citrate, ethanol, water/ethanol mixtures, limonene or other terpenes, isoparaffins such as those known under the trademark Isopar (origin: Exxon Chemical) or glycol ethers and glycol ether esters such as those known under the trademark Dowanol (origin: Dow Chemical Company), or hydrogenated castors oils such as those known under the trademark Cremophor RH 40 (origin: BASF) or mixtures thereof.

As used herein “flavor adjuvant” is meant to refer to an ingredient capable of imparting additional added benefit such as a color (e.g., caramel), chemical stability, and so on. A detailed description of the nature and type of adjuvant commonly used in flavoring compositions cannot be exhaustive. Nevertheless, such adjuvants are well known to a person skilled in the art who will be able to select them on the basis of its general knowledge and according to intended use or application. One may cite as specific non-limiting examples the following: viscosity agents (e.g. emulsifier, thickeners, gelling and/or rheology modifiers, e.g. pectin or agar gum), stabilizing agents (e.g. antioxidant, heat/light and or buffers agents e.g. citric acid), coloring agents (e.g. natural or synthetic or natural extract imparting color), preservatives (e.g. antibacterial or antimicrobial or antifungal agents, e.g. benzoic acid), vitamins and mixtures thereof.

As used herein “perfumery adjuvant” is meant to refer to an ingredient capable of imparting additional added benefit such as a color, a particular light resistance, chemical stability, etc. A detailed description of the nature and type of adjuvant commonly used in perfuming composition cannot be exhaustive, but it has to be mentioned that said ingredients are well known to a person skilled in the art. One may cite as specific non-limiting examples the following: viscosity agents (e.g. surfactants, thickeners, gelling and/or rheology modifiers), stabilizing agents (e.g. preservatives, antioxidant, heat/light and or buffers or chelating agents, such as BHT), coloring agents (e.g. dyes and/or pigments), preservatives (e.g. antibacterial or antimicrobial or antifungal or anti irritant agents), abrasives, skin cooling agents, fixatives, insect repellants, ointments, vitamins and mixtures thereof.

As used herein “personal lubricant” is meant to refer to specialized lubricants, including water-based formulas, that serve to reduce friction with body tissues. In particular, personal lubricants can be used to provide lubrication or slippage during sexual activity. For example, personal lubricants can be used to increase pleasure or reduce pain during sexual intercourse and can help in reducing genital dryness.

As used herein “cooling agent” is meant to refer to compounds that impart a cooling sensation to the skin. Non limited-examples of suitable cooling agent includes WS-23 (2-Isopropyl-N,2,3-trimethylbutyramide), FEMA 3804; WS-3 (N-Ethyl-p-menthane-3-carboxamide), FEMA 3455; WS-5 [Ethyl 3-(p-menthane-3-carboxamido)acetate], FEMA 4309; WS-12 (IR,2S,5R)—N-(4-Methoxyphenyl)-p-menthanecarboxamide, the menthane carboxylic acid esters and amides WS-3, WS-4, WS-5, WS-12, WS-14, WS-30 and mixtures thereof.

Male and female genitalia can be a source of pleasure for individuals and their partners. There are many methods for stimulation and sexual arousal. Among these methods are licking or sucking the genitalia. While this is pleasurable and sexually arousing to the recipient it may be difficult for the one engaging in the licking and sucking because of odors. Refreshing the genitals and the surrounding area can alleviate the odors and can remove any surface germs, thus providing a more pleasurable environment for the partner whose mouth is on and near the genitals.

Personal lubricants are specialized lubricants that serve to reduce friction with body tissues. In particular, personal lubricants can be used to provide lubrication or slippage during sexual activity. For example, personal moistening agents, such as the current invention, can be used to increase pleasure or reduce pain during sexual intercourse, and can help in reducing genital dryness.

Most lubricants generally work by supplying water to a body surface in a gelled or viscous form, including a water-soluble polymer such as a water-soluble cellulose derivative or other water-soluble polymers, such as polyvinylpyrrolidone, polyvinyl alcohol and the like. In use on a body surface, these systems retain a supply of water on the body surface, and the water provides lubrication or slippage. One or more humectants may be added to aid water retention, and thus increase the lubrication provided by the lubricant and/or increase the duration of lubrication.

Typically, personal moistening agents are stored in and dispensed from tubes. Tubes allow the solution to be squeezed from the tube. There are often issues with tubes; they can be messy, it can be difficult to apply the proper pressure to dispense the amount of solution desired, and where is the best place to store the tube.

The present invention forgoes the use of tubes along with the mess and hassle associated with their use, instead a personal moistening agent is carried in a wipe. The personal moistening agent is impregnated within the wipe, and as the wipe is applied to the skin, the moistening agent is deposited on the skin. There are several methods for moistening agent s to be carried by a wipe and deposited on a skin surface. Some of these methods include the polarity of the wipe and the lubricant, the response to heat, and mechanically forcing the moistening agent from the wipe. It is preferred that the wipe be as easy to use as possible, for this it is desirable that the lubricant move from the wipe to the skin without undue effort. A wipe that requires forcing the moistening agent out of the wipe through squeezing or other mechanical force is therefore less desirable. A wipe that requires a swipe across the skin and the moistening agent is deposited is more desirable. One way to accomplish this is to ensure that the wipe is produced without any charge. The moistening agent will have a charge, this is part of the method behind the wipe, and the moistening agent attracts water and keeps it in place on the skin. Therefore, the moistening agent would easily leave the wipe because there is not an attractive force holding it to the wipe.

In one embodiment of the invention, a wipe is provided with a flavoring to accompany the refreshing and lubricating compounds in it. A wipe may be configured to carry a moistening agent compound, a refreshing compound, and a flavoring extract solution. The wipe may be used as an applicator to apply the lubricating compound and flavoring extract to human skin. The pleasant taste of the flavoring solution diminishes the possible objectionable smell and taste when the genitalia (or other body part) is wiped with the wipe. The flavoring extract solution can also be used to moisturize the user's skin.

In some embodiments the solution impregnates the entire wipe. In these embodiments, the wipe includes some or all of; a moisturizing agent, a coloring agent, a flavoring agent and/or a scenting agent. These solutions are formulated to exist together such that none of the included agents will react with any other agent to cause a reaction between the agents or to cause the degradation of any of the agents. This is important because the degradation of the flavoring agent could change the flavor from a pleasant and enjoyable taste to a noxious or unpleasant one.

The combining of all the agents in one wipe has the benefit of making the process of moistening, refreshing, and imparting flavor to the area a single process. The simplicity of the process increases the likelihood that users will utilize the wipes. Furthermore, in these embodiments, the wipes are packaged in a ready to use state. Being in a ready to use state the wipes will be packaged such that they can be used immediately upon removal from the package. This is advantageous for increasing the likelihood of being used as well. Being packaged in a ready to use state also requires packaging that prevents desiccation or drying out of the wipes.

It is noted that the solution may impregnate only a portion of the wipe in other embodiments. For example, specific agents may be provided on portions of each wipe. In some embodiments, the wipe will include; a refreshing agent, a moisturizing agent, a coloring agent, a flavoring agent and/or a scenting agent. In some instances, it is desirable to segregate the different agents from one another. For example, some users want to know that the refreshing step is accomplished before the application of flavor, and the lubrication is applied after the application of flavor. In this way a single wipe could be used in several stages as the encounter progresses. For example, one partner could apply the refreshing agent and then the flavor agent, to then engage in licking or sucking. Following the oral encounter, the moistening portion can be used to aid in intercourse. By way of illustration, half a single wipe may be colored blue to indicate use for refreshing and flavoring (and thus include refreshing and flavoring agents) and the other half of the same wipe may be colored pink to indicate use as a lubricated wipe (and thus include the lubricating agent). Though the wipes in this example utilize two agents for performing separate steps, the wipe will preferably be in a ready to use state similar to that described above. The wipe will be packaged so that it can be removed and used immediately without the addition of any elements.

In some instances, users may desire to have a wipe that is less prone to degradation or loss of effectiveness, these instances utilize a dry compressed wipe and the addition of a wetting agent. Preferably, the wetting agent is water. In these embodiments, the solution is applied to the wipe and released upon the addition of water. Preferably, the solution is dissolved on the material, and the moisture evaporated from off the wipe. Typically, a dry solution is more stable and longer lasting than a solution including moisture, thus the agents in the dry wipes would last longer. Less frequent, or longer periods between uses of the wipes would be reasons users would choose the dry wipes. The dry wipes would be produced similarly to the ready to use wipes, so there would be options such as the all in one wipe, or the wipe with divided portions.

In some embodiments, the solution (or solutions) is added to a specialized wipe. The solution is stored in a vial or a capsule. The solution is added to the material by spraying or pouring for example, from a spray canister or bottle. In yet another example, the solution is emulsified and dispensed as a cream or lotion by a pump dispenser. The emulsified solution may also be dispensed from a squeezable tube having a small hole (or multiple holes in the cap) or provided in a “makeup” type compact for dipping the material into.

The wipe may include a material such as a fiber-based textile with fibers forming an outward raised/embossed texture on a first side and/or second or opposite side. In an example, the solution may be provided in a separate layer. The material is not limited to any particular number of layers. Nor is the material limited to having separate layers. Also incorporated by reference in their entirety are U.S. Patent Publication No. 2002/0127937 to Lange et al., U.S. Patent Publication No. 2006/0151518 to Sarbo et al., U.S. Pat. No. 6,361,784 to Brennan et al. and U.S. Patent Publication No. 2012/0090113 to Manifold.

Also disclosed herein is a method of manufacturing a flavored lubricating wipe. In an example, the method may include providing a material configured for use as a wipe and providing a solution for the material. The solution includes at least a flavoring component.

The flavoring extract can be put into a solution that provides a sufficient amount of flavoring extract per volume of water and with sufficient preservatives so as to resist degradation. For example, degradation can be resisted by making the wipe and/or solution resistant to microbial growth and/or pathogen growth. The amount of flavoring extract added per volume of water (or volume of another non-harmful solution) should be sufficient to provide a pleasant taste.

In accordance with one embodiment, the flavoring solution can be prepared by combining the flavoring extract, so that the flavoring extract contributes 0.1 to 0.5% by weight of the final solution and the water accounts for the remaining percentage by weight.

Preservatives, such as potassium sorbate, sodium benzoate, and decyl glycoside can be added in appropriate amounts to prevent degradation of the flavoring solution.

In another embodiment, a solution can be mixed in the following contributions by weight percentage of the final product (e.g., 92.7% of the weight of the final product could be water).

In some embodiments, the wipes can be prepared by utilizing nonwoven cloth suitable for absorbing solution. A roll of the cloth, preferably with pre-formed lengths of cloth suitable for use as wipes, can then be impregnated with the solution, for example. This is currently well-known by those of ordinary skill in the art. In accordance with one embodiment, the wipe material can be made from biodegradable material so that the wipe will degrade after being disposed of.

Another problem is that some wipes currently available can irritate the skin—especially after repeated wiping over time. This can damage the skin and makes it more sensitive to future wiping.

In some embodiments, moisturizers are used to moisturize and/or soothe the skin at the same time that the skin is being lubricated. Thus, the wet wipe with a moisturizer, is able to both lubricate and moisturize the skin at the same time.

In the embodiment where moisturizing is desired, the amount of moisturizing agent added to the solution can be selected so as to provide a desired moisturizing capability.

In another embodiment, additional additives can be added. For example, as noted above, preservatives may be added to protect the wipe/solution combination from degrading. One example of a preservative is honeysuckle extract. In addition, additives such as vitamin E, chamomile, aloe vera, polysorbate 80, annatto Extract, or natural oils might be added for the properties that those additives provide. For example, orange oil may be added for providing a scent, as well as an antibacterial function. In addition, sugarnate and/or polysorbate 80 can be used as a surfactant. Also, sodium benzoate can be used as a preservative. Green tea extract and aloe vera juice/extract may also be used.

In some embodiments, one might choose to use glycerin in addition to water. Glycerin can be used for its cleaning effect and non-toxic nature if consumed. Glycerin can also provide a moisturizing effect. However, glycerin is believed to have a disagreeable taste to some people. Therefore, it may be necessary to add more flavoring extract than that required for water alone to mask the taste of the glycerin, should glycerin be used. It is noted that preservatives are the bad tasting parts of the formula. Glycerin also has a “soapy” taste. But the addition of the flavoring agent (or other sweeteners discussed herein) imparts a better taste.

In accordance with another embodiment, a wet wipe may be provided with antimicrobial and anti-pathogen properties by using stevia as part of the solution that is imparted on the wet wipe. Stevia has been asserted to exhibit antimicrobial and anti-pathogen properties. Thus, by using it as part of a solution for a wipe product, it is believed that a longer shelf life can be provided for the wipe product. Moreover, this can be accomplished through the use of a natural agent, such as stevia.

To prepare the wet wipes with the stevia solution, one may submerge a roll of wipe material within the stevia solution and allow it to soak for a sufficient time period to allow the solution to be imparted onto or impregnated within the wipe material. Alternatively, the solution may be sprayed or pumped onto the wipe material.

It is noted that sugar or other like sweeteners may leave a sticky residue behind. But the use of stevia (or other sweeteners disclosed herein) can provide the desired taste without leaving behind a sticky residue on the skin. It can provide a soothing and/or moisturizing effect to human skin that counters the effects of a wipe irritating or damaging human skin. And, it can prolong shelf-life by exhibiting antimicrobial and/or anti-pathogen functions. Any of these functions independently or in various combinations can be useful in providing an improved wet wipe product.

According to any one of the above embodiments, the flavored composition may further comprise ingredient imparting a warming, a tingling, a salivating, or an alcohol enhancement effect such as capsicum extract, spice extract (e.g., ginger, maniguette, all types of peppers including Sichuan, piperine, capsaicine, jambu extract, spilanthol.

In a particularly preferred embodiment, the flavor that is put on the wipe is created with 90-100% propylene glycol, 5-10% natural flavor, and less than 5% citric acid. In another preferred embodiment a flavor is used with 40-50% modified corn starch, 40-50% maltodextrin and 5-10% natural flavor. It is also contemplated that a wipe can be made with 0.01-10% sweetener, which could be but is not limited to Stevia, with 0.01-10% fragrance. These amounts are important such that the wipe can leave a residue of flavor and also remain pH balanced for the safety of a woman's genitals. Too much sweetener can create a pH imbalance that can make women prone to UTIs or yeast infections. The amount of sweetener and flavor is important to create the precise balance needed to allow for a desired taste and also be safe for a woman's genitals.

In terms of the physical wipe itself, a particularly preferred embodiment teaches a wipe that is 7″×6″ with a 500% fill rate in a multi-fold four-sided seal individually packaged, made with 70-95% deionized water. The deionized water is important to the invention as the water is clean and contributes to the proper pH of the wipe.

One embodiment calls for a wipe configured to provide lubrication and flavor to an intimate body surface during a sexual encounter, consisting of a material, where the material is a flat sheet with a length measurement of 7″ and a height measurement of 6″, with a 500% fill rate in a multi-fold four sided seal individually packaged The size of the wipe is important so that there is enough liquid to cover enough of the body to allow for the desired flavor. A wipe that is too small will not allow for coverage of enough of the body to make taste meaningful or enjoyable. where the material comprises between 70 and 95% deionized water, where the material is impregnated with a flavoring agent and a moistening or emulsifying agent, where the flavoring agent imparts a pleasant taste to the wipe and where the wipe is a 1-use wipe.

In a preferred embodiment, the flavor that is put on the wipe consists of at least 90% propylene glycol, at least 5% natural flavor, and less than 5% citric acid and 0.01-10% sweetener. The combination of these specific ingredients allows for the flavor to be preserved and contributes to the shelf life of the product. The sweetener could be Stevia or another suitable flavoring. The wipe can also comprise a refreshing agent and a perfume agent, where the flavoring, lubricating, and refreshing agents are in solution in the wipe or applied in a dehydrated manner. The flavoring agent can further comprise a solution for imparting to the surface wiped with the wipe. Because of the chemical makeup for the wipe, it qualifies as Keto.

In another embodiment, the flavor that is put on the wipe can comprise 40-50% modified corn starch, 40-50% maltodextrin and 5-10% of a natural flavor and 0.01-10% of a sweetener.

The invention also contemplates a method of using a single-use wipe to provide lubrication and flavor to an intimate body surface during a sexual encounter, including a first step of opening a wipe package, second step of removing a wipe from the wipe package, applying the wipe to an intimate body surface, and disposing of the wipe, where the wipe can be a flat sheet with a length measurement of 7″ and a height measurement of 6″, with a 500% fill rate in a multi-fold four sided seal individually packaged, where the material comprises between 70 and 95% deionized water. The material can be impregnated with both a scent and a flavoring agent and a lubricating agent, where the flavoring agent imparts a pleasant taste to the wipe, and where the wipe is a 1-use wipe. The method also contemplates that the material may further comprise a refreshing agent and a perfume agent, where the flavoring, lubricating, and refreshing agents are in solution in the wipe, where the flavoring agent further comprises a solution for imparting to the surface wiped with the wipe.

This method contemplates that the flavor that is put on the wipe consists of 40-50% modified corn starch, 40-50% maltodextrin and 5-10% of a natural flavor and 0.01-10% of a sweetener, or, alternatively, where the flavor that is put on the wipe consists of at least 90% propylene glycol, at least 5% natural flavor, and less than 5% citric acid and 0.01-10% sweetener.

A final product contemplates deionized water as a solvent, with sodium benzoate (granular) and potassium sorbate nutrinova as preservatives, with a flavor of natural juicy peach type flavor powder PH-63439, a humectant of glycerin 99.7% USP Kosher, a viscosity agent of xanthan gum FN (food grade) and a PH adjustor of citric acid Anhy USP/FCC GPD.

It is understood that a person skilled in the art is perfectly able to design optimal formulations for the desired effect by admixing the above-mentioned components of a flavoring or perfuming composition, simply by applying the standard knowledge of the art as well as by trial-and-error methodologies.

For the sake of clarity, it is also understood that any mixture resulting directly from a chemical synthesis, e.g. a reaction medium without an adequate purification, in which the compounds of the invention would be involved as a starting, intermediate or end-product could not be considered as a flavoring or perfuming composition according to the invention as far as said mixture does not provide the inventive compound in a suitable form for perfumery or for flavor. Thus, unpurified reaction mixtures are generally excluded from the present invention unless otherwise specified.

All patents and published patent applications referred to herein are incorporated herein by reference. However, any reference to prior publication is not, and should not be taken as an acknowledgement, admission, or suggestion that the prior publication, or any information derived from it is part of the general common knowledge in the field of endeavor to which this specification relates. The invention has been described with reference to various specific and preferred embodiments and techniques. Nevertheless, it is understood that many variations and modifications may be made while remaining within the spirit and scope of the invention.

What is claimed is:

1. A wipe configured to provide moisturization and flavor to an intimate body surface during a sexual encounter, consisting of:
a material, where the material is a flat sheet with a length measurement of 7″ and a height measurement of 6″, with a 500% fill rate in a multi-fold four-sided seal individually packaged, where the material comprises between 70 and 95% deionized water,
where the material is impregnated with a flavoring agent and a moisturizing agent, where the flavoring agent imparts a pleasant taste to the wipe, and where the wipe is a 1-use wipe.

2. The wipe of claim 1, where the flavoring agent that is put on the wipe consists of at least 90% propylene glycol, at least 5% natural flavor, and less than 5% citric acid and 0.01-10% sweetener.

3. The wipe of claim 2, where the sweetener is Stevia.

4. The wipe of claim 3, further comprising a refreshing agent and a perfume agent, where the flavoring, moisturizing, and refreshing agents are in a solution in the wipe.

5. The wipe of claim 4, where the flavoring agent further comprises a solution for imparting the flavoring agent to a surface wiped with the wipe.

6. The wipe of claim 1, where the flavoring agent that is put on the wipe consists of 40-50% modified corn starch, 40-50% maltodextrin and 5-10% of a natural flavor and 0.01-10% of a sweetener.

7. The wipe of claim 6, where the sweetener is Stevia.

8. The wipe of claim 7, further comprising a refreshing agent and a perfume agent, where the flavoring, moisturizing, and refreshing agents are in solution in the wipe.

9. The wipe of claim 8, where the flavoring agent further comprises a solution for imparting to the surface wiped with the wipe.

10. The wipe of claim 1, were the wipe additionally comprises a solvent, where the solvent is deionized water, two preservatives, where the two preservatives are sodium benzoate (granular) and potassium sorbate nutrinova, a flavor, where the flavor is natural juicy peach type flavor powder PH-63439, a humectant, where the humectant is glycerin 99.7% USP Kosher, a viscosity agent, where the viscosity agent is xanthan gum FN (food grade) and a PH adjustor, where the PH adjustor is citric acid Anhy USP/FCC GPD.

11. A wipe configured to provide moisturizing and flavor to an intimate body surface during a sexual encounter, comprising;
a material,
where the material is a flat sheet with a length measurement of 7″ and a height measurement of 6″, with a 500% fill rate in a multi-fold four-sided seal individually packaged, where the material comprises between 70 and 95% deionized water, where the material is impregnated with a flavoring agent; and a lubricating agent,
wherein the flavoring agent imparts a pleasant taste to the wipe.

12. The wipe of claim 11, where the flavoring agent that is put on the wipe comprises between 90% and 100% propylene glycol, between 5% and 10% of a natural flavor, and less than 5% citric acid and 0.01-10% sweetener.

13. The wipe of claim 12, where the sweetener is Stevia.

14. The wipe of claim 13, further comprising a refreshing agent and a perfume agent, where the flavoring, moisturizing, and refreshing agents are in solution in the wipe.

15. The wipe of claim 14, where the flavoring agent further comprises a solution for imparting to the surface wiped with the wipe.

16. The wipe of claim 11, where the flavoring agent that is put on the wipe consists of 40-50% modified corn starch, 40-50% maltodextrin and 5-10% natural flavor and 0.01-10% sweetener.

17. The wipe of claim 16, where the sweetener is Stevia.

18. The wipe of claim 17, further comprising a refreshing agent and a perfume agent, where the flavoring, lubricating, and refreshing agents are in solution in the wipe, where the flavoring agent further comprises a solution for imparting to the surface wiped with the wipe.

19. The wipe of claim 11, were the wipe additionally comprises a solvent, where the solvent is deionized water, two preservatives, where the two preservatives are sodium benzoate (granular) and potassium sorbate nutrinova, a flavor, where the flavor is natural juicy peach type flavor powder PH-63439, a humectant, where the humectant is glycerin 99.7% USP Kosher, a viscosity agent, where the viscosity agent is xanthan gum FN (food grade) and a PH adjustor, where the PH adjustor is citric acid Anhy USP/FCC GPD.

20. A method of using a single-use wipe to provide moisturizing and flavor to an intimate body surface during a sexual encounter,
including a first step of opening a wipe package, second step of removing a wipe from the wipe package, applying the wipe to an intimate body surface, and disposing of the wipe,
where the wipe consists of
a material,
where the material is a flat sheet with a length measurement of 7″ and a height measurement of 6″, with a 500% fill rate in a multi-fold four-sided seal individually packaged, where the material comprises between 70 and 95% deionized water,
where the material is impregnated with a flavoring agent and a moisturizing agent, where the flavoring agent imparts a pleasant taste to the wipe, and where the wipe is a 1-use wipe,
further comprising a refreshing agent and a perfume agent, where the flavoring, lubricating, and refreshing agents are in solution in the wipe,
where the flavoring agent further comprises a solution for imparting the flavoring agent to the surface wiped with the wipe.

21. The method of claim 20, where the flavoring agent that is put on the wipe consists of 40-50% modified corn starch, 40-50% maltodextrin and 5-10% of a natural flavor and 0.01-10% of a sweetener.

22. The method of claim 20, where the flavoring agent that is put on the wipe consists of at least 90% propylene glycol, at least 5% natural flavor, and less than 5% citric acid and 0.01-10% sweetener.

23. The method of claim 20, were the wipe additionally comprises a solvent, where the solvent is deionized water, two preservatives, where the two preservatives are sodium benzoate (granular) and potassium sorbate nutrinova, a flavor, where the flavor is natural juicy peach type flavor powder PH-63439, a humectant, where the humectant is glycerin 99.7% USP Kosher, a viscosity agent, where the viscosity agent is xanthan gum FN (food grade) and a PH adjustor, where the PH adjustor is citric acid Anhy USP/FCC GPD.
